(19) United States
(12) Patent Application Publication
(10) Pub. No.: US 2024/0091448 A1
(43) Pub. Date: Mar. 21, 2024

(54) MEDICAMENT DELIVERY DEVICE

(71) Applicant: SHL MEDICAL AG, ZUG (CH)

(72) Inventors: Anders Boström, Ingaro (SE); Abhilash Joseph, Nacka Strand (SE)

(21) Appl. No.: 18/267,216

(22) PCT Filed: Dec. 9, 2021
(86) PCT No.: PCT/EP2021/084912

§ 371 Date: Jun. 14, 2023

(30) Foreign Application Priority Data
Dec. 24, 2020 (EP) 20217176.5

(51) Int. Cl. A61M 5/20; A61M 5/315; A61M 5/32

(52) U.S. Cl. CPC A61M 5/2033; A61M 5/31571; A61M 5/3245; A61M 2005/2073; A61M 2005/3142

(57) ABSTRACT

The disclosure describes a medicament delivery device having an actuation mechanism configured to act on a medicament container for expelling a medicament, where mechanism has a rear assembly configured to hold said actuation mechanism in a pre-tensioned state, wherein the rear assembly further comprises a transport lock mechanism a support and holding mechanism for a feedback member and activation member an activation member guard. Further an elongated housing having a proximal end and an opposite distal end, accommodating the rear assembly, the medicament container, the actuation mechanism and the activation member guard, wherein the housing further comprises guiding members able to interact with the transport lock mechanism of the rear assembly and/or a container holder for the medicament container. A biased activation member guard is axially slidable with respect to the housing from a proximal extended position to a retracted position and operably connected to a tubular coupling member.

### TECHNICAL FIELD

The present invention relates to an improved medicament delivery device, easier to secure and assemble automatic medicament delivery device, in particular an autoinjector for dispensing a fluid product. Automatic medicament delivery devices are known in the prior art, these still have several drawbacks which are to be overcome. The present invention thus aims to overcome these drawbacks and to provide an improved secure transport locking mechanism, an improved drive mechanism, an improved syringe support device, an improved cap assembly and an automatic shielding and locking mechanism of an injection needle.

### BACKGROUND

Medicament delivery devices such as auto-injectors are very common on the market as they facilitate for users to manage medicament administration.

For example, medicament delivery devices such as those described in WO 2011/123024 for example have already been very commercially successful due to a combination of properties such as robustness, simplicity and usability. Nevertheless, the applicant has appreciated that there is still further scope for improvement of the drive mechanism, the locking features, and other features in medicament delivery devices such as those described in WO 2011/123034 or WO 2015/169608 or WO 2016/169719 for example.

The present disclosure is applicable to several medicament delivery devices, including, but not limited to, devices that automatically, semi-automatically or manually deliver one or more doses of medicament through injection (needle and needleless), inhalation, infusion, atomization, drops, patches, and implants. Incorporating one or more automatic feedback mechanisms into these medical devices ensures that the user of the device will be notified of the beginning of the medicament delivery sequence.

One aspect of the present disclosure relates to medicament delivery devices that are delivered as sub-assemblies for final assembly into, for instance, auto-injectors. One of the sub-assemblies may be a power pack, which may comprise a pre-tensioned plunger rod, arranged with a control member that is configured to release the pre-tensioned plunger rod in the assembled medicament delivery device. For this reason, the control member is movable, so that another element, typically comprised in a different sub-assembly, may interact with the control member when the user of the medicament delivery device intends to administer a dose of medicament. An accidental activation of the power packs during the transport of the sub-assemblies, due to vibrations, movements and impacts shall be prevented without complicating or impeding the assembly or locking mechanism. It is therefore necessary to ensure that the control members are held securely so that they do not accidentally activate the power packs. Some attempts have been made to solve these problems. WO2018/206583 discloses a transport lock assembly for a medicament delivery device, wherein the transport lock assembly comprises a power pack having a locking member configured to interact with a control member, which locking member is movable, relative to the control member, from a first state in which the control member is immobilized, to a second state in which the control member is free to move; wherein the transport lock assembly further comprises a housing part having a key member, such that assembly of the powerpack with the housing part causes the key member to move the locking member from the first state to the second state.

One aspect of the disclosure relates to a drive mechanism, which often are assembled with the power source, in many cases a compression drive spring, in a tensioned state. The drive springs are often held in this tensioned state by components of the unit and do not in that non-active state exert any force on the housing of the medicament delivery device.

When the components are assembled inside the housing, it is often enclosed by some sort of enclosure cap, such as an end cap. The attachment may be performed in many ways such as gluing, plastic welding, screw threads or bayonet threads. Some of these attachment means often provide a secure attachment, but are not optimal from an assembly point of view since they either take quite an amount of time to perform an attachment, or they make the moulding complicated in order to get the form and the required tolerances, which increases the manufacturing costs. This may not be acceptable if for example the medicament delivery device is a so-called disposable that is thrown or discarded after use.

Another prior art document, U.S. Pat. No. 5,026,349A describes a medical injector apparatus including a syringe, having a barrel with a lateral projection, a needle, a plunger in the barrel having a portion extending from the barrel opposite the needle. The injector apparatus has a body having a forward end for the needle and a rearward end portion of the extending portion of the plunger; a trigger rearwardly movable from in front of the body by pressure from the flesh of a user; a receiver for holding the syringe axially aligned with the body, including a guide having a cavity for releasably holding the barrel and having a lateral opening for radial movement of the barrel into the cavity; an aligner holding the syringe in lateral alignment with the body during insertion of the syringe into the receiver, including opposite entry surfaces at a forward extremity of the guide for laterally centering a contacted portion of the barrel, and a rear guide located rearwardly of the entry surfaces for laterally centering a rear portion of the syringe; the syringe being movable in the body from a first position forwardly to a second position, the needle projecting beyond the trigger surface in the second position for piercing the flesh; an actuator for forwardly moving the plunger; a catch for preventing operation of the actuator; and a releaser connected to the trigger for releasing the catch, whereby the actuator moves the plunger forwardly in the barrel, the syringe being moved from the first position to the second position during an injection.

One aspect of the disclosure relates to an automatic shielding mechanism of an injection needle after the medicament delivery device has been withdrawn. This is often performed by a needle guard that extends in the proximal direction by a spring or the like when the medicament delivery device is removed from the dose delivery site and surrounds the needle. In a fully extended position, the needle guard is locked by appropriate locking elements so that the needle guard cannot be pushed back into the medicament delivery device again, exposing the injection needle. These functions may be triggered or activated by the movement of the needle guard during retracting into the medicament delivery device as well as extending out of the medicament delivery device. One example of these features is disclosed in document WO2016/202555 which relates to a medicament delivery device comprising a housing, a biased medicament delivery member guard arranged movable in relation to the housing from a proximally extended position to a retracted position, a power pack comprising a plunger rod and a force element arranged to act on said plunger rod, which power pack is actuated by a release clip movable in relation to the housing and arranged to said plunger rod for releasably holding said plunger rod with the force element in a tensioned state. A rotator is arranged with a guide element and a medicament delivery member guard lock element on its outer surface, wherein said medicament delivery member guard is arranged with release clip activation elements for releasing said plunger rod. A rotator activator element cooperates with the guide element for turning the rotator. Upon movement of the medicament delivery member guard to the retracted position, and upon movement of said medicament delivery member guard back to the extended position, the rotator activator element will engage with the medicament delivery member guard lock element and lock the medicament delivery member guard in a shielding position after removal of the medicament delivery device from a medicament delivery site.

One aspect of the disclosure relates to an administration mechanism as described in the prior art WO 2018/010947, which relates to an administration mechanism for a medicament delivery device, comprising: a movable sleeve, and a delivery member cover having a distal end portion configured to receive the movable sleeve, and configured to be linearly displaceable between a first position and a second position relative to the movable sleeve, wherein the distal end portion has an end face defining a guide surface and the movable sleeve has a radially outwards extending first follower structure configured to cooperate with the guide surface when the delivery member cover is moved from the first position towards the second position, causing the movable sleeve to rotate.

One aspect of the disclosure relates to a syringe holder of an autoinjector device for use in combination with a syringe, as described in the prior art WO2013/089620 which describes an autoinjector device comprising a movable plunger, a needle and a tubular needle shield. The autoinjector device has an elongated housing having a drive mechanism and a syringe holder in order to support the fragile syringe and to prevent damage to the syringe. It also relates to a method for assembly of the autoinjector device according to the invention. Further solutions described in the prior for example in WO 2013/077800 which describes an invention related to an Injection device comprising a housing, a container holder being configured for accommodating a medicament container having a needle attached to one end thereof and a stopper sealing and slidable arranged inside the medicament container at the other end. A drive unit comprising a plunger rod and plunger drive means, wherein the plunger drive means is operationally associated with said drive unit, a first energy accumulating member, and an injection indication mechanism, wherein the injection indication mechanism comprises a tactile signalling element and a drive mechanism for driving said tactile signalling member, said drive mechanism being coupled to said plunger drive means.

Another example of prior art solution is given in U.S. Pat. No. 8,961,463 B2, describing a two-dose autoinjector for a medicament wherein the locking and releasing of the drive spring of the autoinjector is controlled through stepped guides with ramps for two successive sliding of slides operated by the spring and connected with the Syringe and plunger. The guides and the slides are pivotable relative to one another and the sliding direction, while the syringe can only slide axially. To enable or disable the sliding of the slides within the guides an angularly mobile arming member is provided formed with a guide track Substantially equal to that of the stationary member where the guides are formed. However, misalignment during final assembly leading to failures may not be prevented with these features.

One aspect of the disclosure relates to a cap assembly for a medicament delivery device. The cap assembly is configured to be mounted to a medicament delivery member shield thereby protecting the medicament delivery member shield and the medicament delivery member. In order to protect and to keep the medicament delivery member sterile, the medicament delivery member may be provided with a medicament delivery member shield, or sheath, such as a Flexible Needle Shield (FNS) or a Rigid Needle Shield (RNS). The medicament delivery member shield may thus be attached to the medicament container to cover the medicament delivery member, during assembly of the medicament container or of the medicament delivery device. Moreover, the medicament delivery device may comprise a removable cap which is mounted to the proximal end of the housing, i.e. that end which is placed towards the injection site during medicament delivery, of the medicament delivery device, or to the proximal end of the medicament container. The removable cap has the function of providing mechanical protection of the medicament delivery member while attached to the housing or medicament container, and to remove the medicament delivery member shield when the cap is removed from the housing.

### BRIEF DESCRIPTION

The present disclosure is applicable to several medical devices, including, but not limited to, devices that automatically, semi-automatically or manually deliver one or more doses of medicament through injection (needle and needleless), inhalation, infusion, atomization, drops, patches, and implants. Incorporating one or more automatic feedback mechanisms into these medical devices ensures that the user of the device will be notified of the beginning of the medicament delivery sequence.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

During the final assembly and prior the transport of the sub-assemblies, medicament delivery devices especially engaging part can be damaged or weekend, transport lock mechanisms and assembly mechanisms have to be strong enough to keep the sub-assemblies safely together during transport and final assembly and simple enough to be easily assembled. In view of this a general objective of the present disclosure is to provide a medicament delivery device which solves or at least mitigates the problems of the prior art.

According to a first aspect, the medicament delivery device comprises an actuation mechanism configured to act on a medicament container for expelling a medicament comprising a rear assembly configured to hold said actuation mechanism in a pre-tensioned state, wherein the rear assembly further comprises a transport lock mechanism a support and holding mechanism for a feedback member and activation member an activation member guard; further an elongated housing having a proximal end and an opposite distal end, accommodating the rear assembly, the medicament container, the actuation mechanism and the activation member guard, wherein the housing further comprises guiding members able to interact with the transport lock mechanism of the rear assembly and/or a container holder for the medicament container; and a detachable rear cap; wherein the biased activation member guard axially slidable with respect to the housing from a proximal extended position to a retracted position and operably connected to a tubular coupling member, rotating the coupling member from an initial non-activated rotational position to an activated rotational position when the activation member guard slides from the extended position to the retracted position; and wherein the coupling member further comprises a blocking element arranged to block the activation member guard in a final position after expelling of the medicament.

According to a further aspect the activation member guard further comprises at least one arm with a flexible radially inwardly extending ledge arranged in a recess of the arm of the activation member guard and excreting a force on a medicament holder.

In a further aspect of the medicament delivery device the inwardly extending ledge acts as stopper when the medicament holder has been introduced from the distal end into the distal opening of the activation member guard, as the distal end of the medicament holder surpasses the ledge, which may flex back into a radially inward position, such that the medicament holder being prevented from moving back towards the distal end.

The inwardly extending ledge, further comprises a radially inwardly extending protrusion providing an enlarged stopping surface for the medicament holder, tolerating less flexing movement of the ledge or a radially smaller medicament holder.

The transport lock mechanism comprises a radially outwardly extending locking member arranged on an actuator and configured to interact with a mating member being a part of the coupling member and configured to engage with the locking member such that the coupling member is prevented of axial movement.

The transport lock mechanism when in a first state, the mating member mates with the corresponding locking member of the coupling member such that rotation of the coupling member relative to the actuator and the locking member is prevented; in a second state, the guiding members of the housing abut a surface of the transport lock mechanism and during final assembly pushes the locking member out of the engagement with the corresponding mating member.

According to a further aspect the housing comprises at least one axially extending protrusion being guiding members positioned such that at least one arm of the activation member guard is axially guided and a radial movement beyond the abutment with the protrusion is prevented by the protrusion.

According to a further aspect, the actuation mechanism further comprises a resilient member and wherein the activation member comprises at least one arm with a tapered distal portion wherein the tapered distal portion defines a guide surface at one of the longitudinal sides, wherein the guide surface is configured to cooperate with the coupling member, when the resilient member biases the activation member guard towards the activated rotational position.

The rotating the coupling member from the initial non-activated rotational position to the activated rotational position comprises the guide surface abutting a radially outwards extending protrusion optionally on a circumferential annular rib on the coupling member, wherein the protrusion optionally has a to the guide surface complementary oblique contact surface.

The coupling member further comprises a protrusion defining a flexible ledge being compressible radially inwards, when one of the longitudinal sides of the activation member arm slides above the flexible ledge.

The flexible ledge is a blocking element which flexes radially outwards when a first resilient member of the rear assembly actuates the expelling of the medicament and thereby displaces the activation member guard axially in a proximal direction, wherein the flexible ledge flexes radially outward when a distal end portion of the activation member arm disengages and releases the flexible ledge; wherein the flexible ledge then provides an abutting surface for the distal end portion blocking a distal movement of the activation member guard.

The medicament delivery device further comprises guiding members to control the movement of the container holder within the housing when the container holder is longitudinally mounted within the housing, wherein the guiding members define protrusions arranged along the inner surface of the housing configured to fit into corresponding groves being guiding members arranged along the outer surface of the container holder.

The guiding members of the housing further are abutting the surface of the transport lock mechanism and during final assembly pushes the locking member out of the engagement with the corresponding mating member.

The support and holding mechanism for the feedback member and activation member comprising the actuator having one or more engagement means configured to engage with engagement members of the rear cap, wherein the engagement member has a flexible ledge with an oblique proximal edge configured to guide the flexible ledge into the housing during assemble.

The engagement means define a recess on at least one longitudinal protrusion arranged along the outer surface of the actuator, when the rear cap being assembled, the engagement member has a complementary fitting shape to fit into the recess and securely hold the cap in place, wherein the recess optionally has a hook for holding the engagement member of the cap locked such that once assembled the cap is not removable.

The actuator further comprises at least one locking element in form of a radially inwardly extending protrusion along the inner surface of the actuator, the locking element is abutting the bottom of a supporting recess formed by two lateral arms of a distal end portion of the support structure, wherein the locking element engages with by fitting within the width of the recess in order to prevent the feedback element to move distally and prevent a rotational movement of the support structure.

The actuator further comprises at least one engagement member in form of radially inwardly extending protrusion configured to engage with a corresponding recess arranged on the outer surface of the support structure, during assembly the support structure slides from the proximal end in a distal direction within the tubular actuator until the engagement member flexes into the corresponding recesses preventing any further movement of the support structure in a distal direction.

### BRIEF DESCRIPTION OF THE DRAWINGS

Embodiments of the invention will now be described by way of example only and with reference to the accompanying drawings, in which:

FIG. 1 shows a perspective view of an assembled medicament delivery device according to the present disclosure.

FIG. 2 illustrates a partially exploded view of the assembled medicament delivery device of FIG. 1.

FIG. 3 shows an exploded view of the delivery device of FIG. 1.

FIG. 4A and FIG. 4B showing the medicament device in a prior to use status with a perspective view on the actuation mechanism without the housing in FIG. 4A and with the housing in 4B.

FIG. 5A and FIG. 5B showing the medicament device in an activated state ready to use with a perspective view on the actuation mechanism without the housing in FIG. 5A and with the housing in 5B.

FIG. 6A and FIG. 6B showing the medicament device in a after use status with a perspective view on the actuation mechanism without the housing in FIG. 6A and with the housing in 6B.

FIG. 7A depicts the sub-assembly of the power pack with the medicament container.

FIG. 7B shows a perspective view of the power pack.

FIG. 7C shows a fully exploded view of the power pack of FIG. 7B.

FIG. 7D shows the support structure of the power pack in further detail.

FIG. 7E depicts a perspective view of the activation member of the power pack of FIG. 7B.

FIG. 8A shows a cross sectional view of the housing along the longitudinal axis L.

FIG. 8B shows another cross-sectional view of the housing of FIG. 8A rotated by 180 degrees.

FIG. 8C shows a perspective view of the housing.

FIG. 8D shows a side view of the sub assembly of FIG. 4A before the locking mechanism being activated.

FIG. 9A to 9C are illustrating the two stages activation phase and use and ready to use. 9A shows the needle cover position before activation. FIG. 9B shows the needle cover displacement during activation and FIG. 9C shows the needle cover position in an activated state.

FIG. 10A to 10D show the displacement of the needle cover of FIG. 9A to 9C in perspective views with further details. FIG. 10E showing the locking position of the needle cover after use.

FIG. 11A, FIG. 11B and FIG. 11D show perspective details of the transport locking mechanism of the power pack.

FIG. 11C shows a perspective view of the housing.

FIG. 12A show a detail of the distal part of the support structure of the power pack.

FIG. 12B, FIG. 12C and FIG. 12D show the rear cap in different perspective views.

FIG. 12E and FIG. 12F show a different rear assembly with the rear cap, FIG. 12E shows a perspective view.

FIG. 12F shows a cross sectional view of the embodiment of FIG. 12E

FIG. 12G shows an alternative embodiment of the rear assembly.

FIG. 12H shows a partially exploded view of the rear assembly of FIG. 12G.

FIG. 12J and FIG. 12K show a perspective and side view of the rear assembly of FIG. 12G without the cap attached.

FIG. 12L and FIG. 12M show a perspective view of the rear cap of FIG. 12G.

FIG. 13A cross sectional view of the rear support structure and the actuator as assembled.

FIG. 13B perspective view of the actuator.

FIG. 14 shows the actuator and the support structure in a side view.

### DETAILED DESCRIPTION

Various modifications to the embodiments described are possible and will occur to those skilled in the art without departing from the invention which is defined by the following claims.

In general, the medicament delivery device may be an auto-injector or a pen injector, for example. In one example, the medicament delivery device comprises a housing 10, a medicament container 20 inside the housing, a container holder 30, a medicament delivery actuation mechanism 11, and a removable protective cap 16, wherein the medicament container comprises a medicament barrel, a syringe, a bag, a cartridge or any appropriate medicament container, a medicament delivery member such as a needle or nozzle and a medicament delivery member shield 38, and wherein the cap 16 comprises a needle shield remover 160. The medicament delivery device may be a single use or a multiple use device.

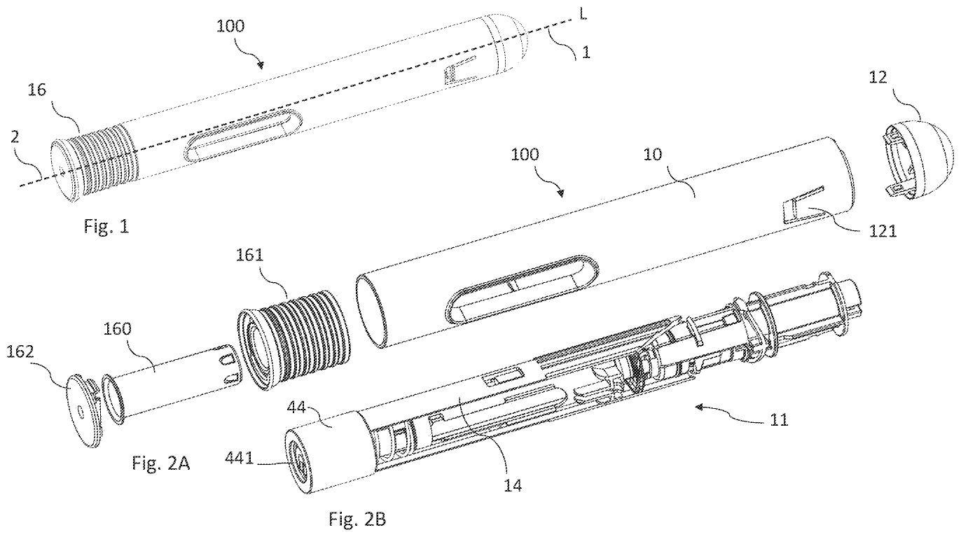

The embodiment of the medicament delivery device 100 of the present disclosure shown in the drawings is designed with a generally elongated, tubular housing 10 that can have a generally rounded shape or any other suitable cross-sectional shape. The medicament delivery device 100 has a distal end 1 and a proximal end 2 extending along a longitudinal axis L, as shown in FIG. 1. The housing 10 is arranged to accommodate the medicament delivery actuation mechanism 11, shown in FIG. 2. At the proximal end 2 of the housing 10, the medicament delivery device 100 may further be provided with the removable protective cap 16. In the case that the container has a fixedly attached injection needle with a needle shield, the cap 16 may also be provided with a needle shield remover, such that the needle shield is removed from the needle when the cap 16 is removed from the proximal end of the housing 10. The protective cap 16 is designed to be engaged and form a removable connection with the proximal part of the housing. The protective cap 16 comprises a generally tubular body 161 provided with optionally a circumferential outwardly extending ledge, and a top cover 162 intended to facilitate the gripping of the protective cap 16. In general, the protective cap has a generally similar cross-sectional shape as the housing 10 and has a distally directed opening. The tubular body 161 of the protective cap 16 often has a diameter equal too, or slightly larger than the outer diameter of the medicament delivery member shield remover 160.

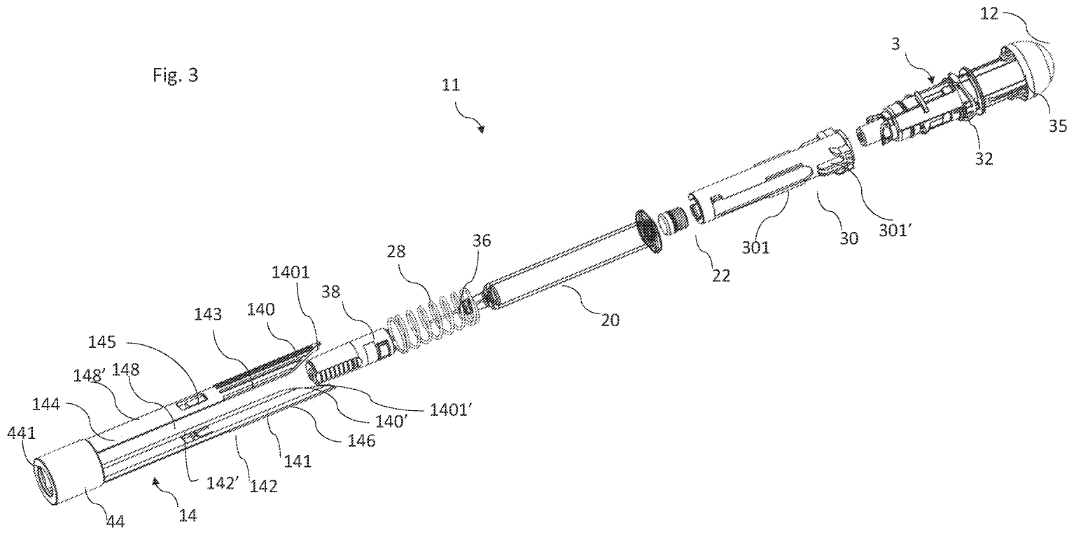

The medicament container 20 further comprises a medicament delivery member cover or shield 38, in the form of a generally elongated tubular member. The protective cap 16 is connected to the proximal end of the medicament delivery actuation mechanism 11 by its central element, i.e. the medicament delivery member shield remover 160, which is extending into a tubular activation member 14 i.e. a needle cover and surrounding a medicament delivery member shield 38 as it is shown in FIG. 3.

The medicament delivery actuation mechanism 11 as shown in FIG. 2B is capable of, upon activation, effecting a delivery of a dose of liquid medicament from the medicament container 20 arranged inside the housing 10 via a medicament delivery member 36, such as an injection needle, which is protected and covered by the delivery member shield 38, for example a needle shield. The medicament delivery member shield 38, as shown, is a so called RNS (rigid needle shield) keeping the injection needle 36 in a sterile environment until the medicament delivery device 100 is ready for use. Alternatively, also a FNS (flexible needle shield) can be used as the medicament delivery member shield. This shield 38 is removed from medicament container 20 by a medicament delivery member shield remover 160, which is axially fixed to protective cap 16 and has a generally tubular body having an inner diameter generally corresponding to the outer diameter of the medicament delivery shield 38. The medicament delivery actuation mechanism 11 may have a variety of designs and functions that are applicable and functional with the locking mechanisms disclosed in the present disclosure.

The medicament container 20 is further arranged within the container holder 30 to retain the medicament container within the housing 10. The container holder 30 optionally further has guiding members in form of longitudinal protrusions for example ribs 301, 301′ on the outer shell of the container holder 30, configured to engage with the receiving structures and provide an aligning feature and or stopping or holding feature during or after assembly. The medicament container 20 has a predetermined volume of medicament and a slidable stopper 22 sealing the distal end of the medicament container 20 as shown in FIG. 3. At the proximal end of the medicament container 20 the delivery member 36 is fixedly or removably attached. The medicament container 20 may be a syringe provided with a needle 36 as the delivery member, but not be limited to this. Other embodiments could include a medicament cartridge having a membrane, or the like, where a delivery member can be other than a needle, for example a nozzle or a mouthpiece.

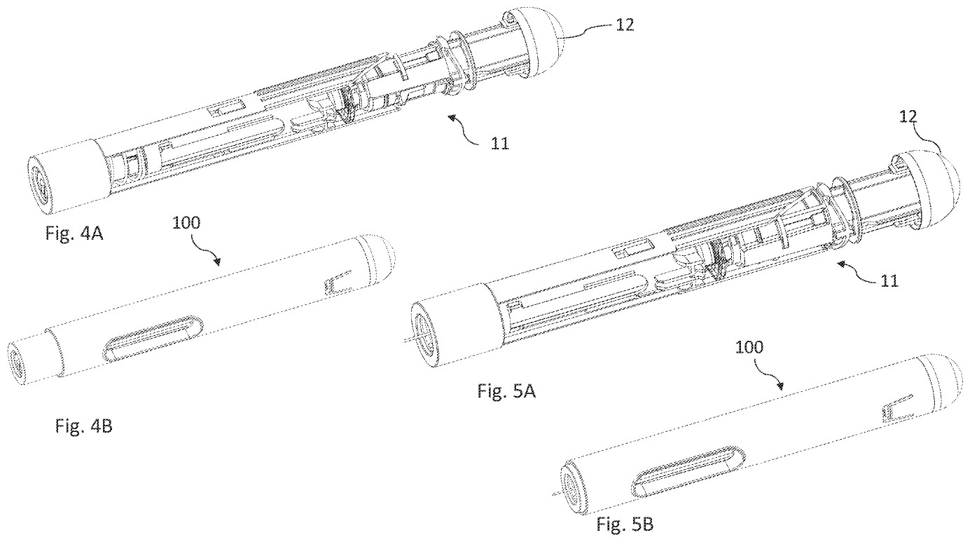

In one embodiment the delivery actuation mechanism 11 comprises the activation member 14 having a generally hollow tubular body with an annular contact member 44 at its proximal end 2, and a first resilient member 28 which is arranged between a distal facing circular ledge of the contact member and proximal facing surface of the container holder 30. The first resilient member 28 is configured to exert a force on the activation member 14 in a proximal direction. The contact member 44 is aligned with the proximal end 2 of the housing 10 when the medicament delivery device 100 is in an activated state, and a portion of the tubular body with the contact member 44 is extending out of the proximal end of the housing 10 at a predetermined distance from the proximal end 2 of the housing 10, when the medicament delivery device is in a ready-to-use state as shown in Figures FIG. 4A and FIG. 4B.

The tubular activation member 14 is movably arranged at the proximal end 2 of the housing 10, and is movable between and extended position, as shown in FIG. 4A, 4B, covering the delivery member 36 for example a needle, and a retracted position, as shown in FIG. 5A, 5B, wherein a penetration may be performed with the needle 36. The activation member 14 is preferably urged in the proximal direction by the first resilient member or spring 28. The activation member 14 also serves to protect and conceal the medicament delivery member 36 being for example a needle.

The delivery actuation mechanism 11 further comprises a rear assembly 3 or power pack comprising an elongated guide rod 26, for example a plunger rod an actuator 35 or rotator, a second resilient member or compression spring 24, a plunger rod 34 that is arranged to act on the stopper 22 for delivering a dose of medicament through the needle 36. The plunger rod 34 a hollow rod, driven by the second resilient member for example a compression spring 24.

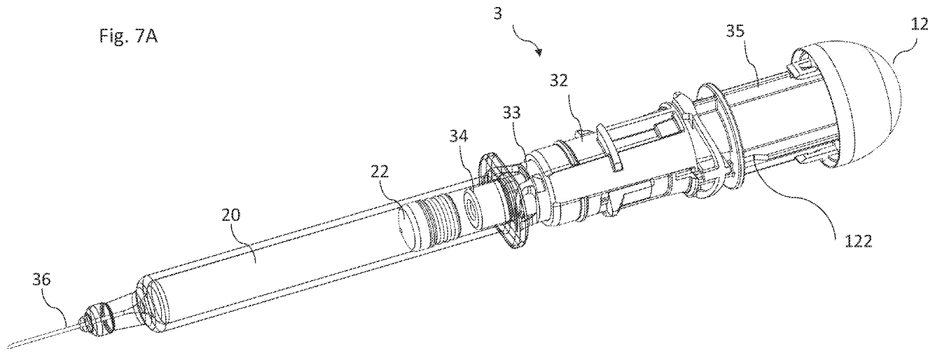

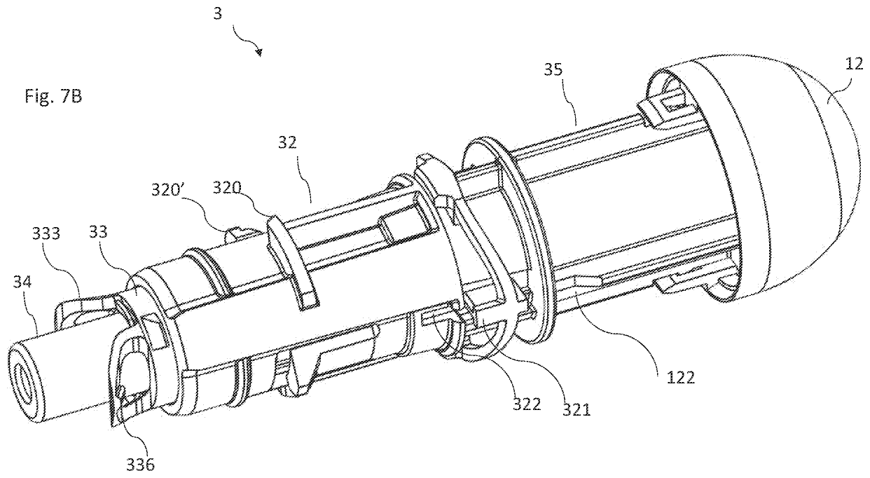

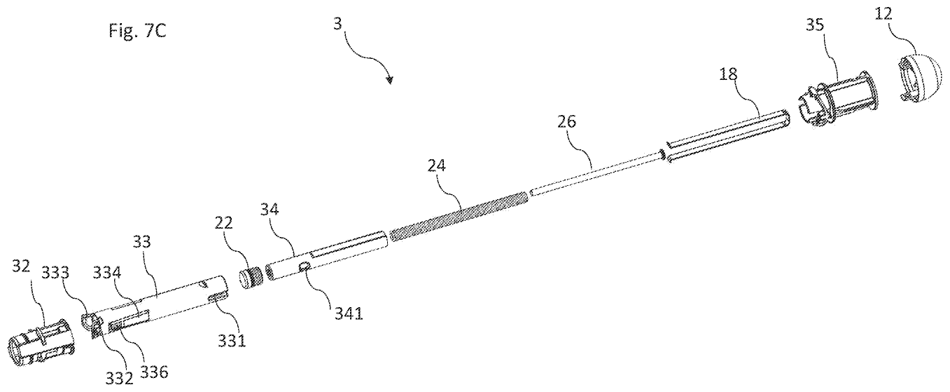

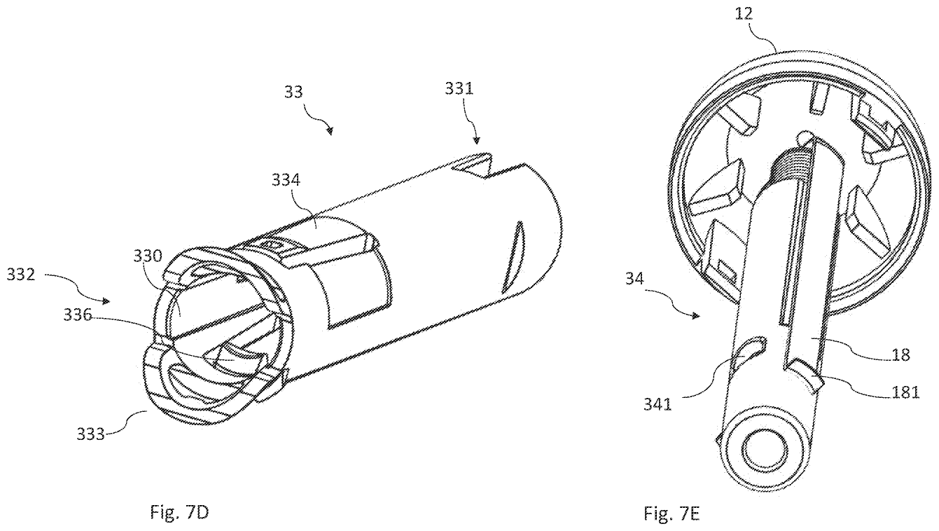

FIG. 3 shows an exploded view of the actuation mechanism 11 of the medicament delivery device 100, and FIG. 7C shows an exploded view of the rear assembly 3, which furthermore comprises a support structure 33 for holding the plunger rod 34 in a pre-tensioned state, a movable coupling member 32 configured to interact with the support structure for releasing the plunger rod, and a feedback member 18 all being a part of the rear assembly, i.e. the power pack 3. The support structure has an elongated tubular body extending coaxial with the plunger rod 34 from a distal end 331 to a proximal end 332, The support structure 33 comprises at its proximal end 332 holding elements 334 in the form of arms that are flexible in the generally radial direction as shown in FIG. 7D. The free ends of the arms 334 are arranged with radially inwardly extending ledges 336, which ledges 336 are arranged to fit into recesses 341 of the plunger rod 34, for releasably holding the plunger rod 34 as shown in FIG. 7E. The free ends of the arms 334 are further arranged with proximally extending and radially outwardly directed support members 333, forming arc shaped support members, configured to interact with the distal end surface of the medicament container. The inner surface of the proximal part of the support structure 33 is arranged with longitudinally extending grooves 330 illustrated in FIG. 7D, which is configured to interact with outwardly extending ledges 181 of the feedback member 18 as shown in FIG. 7E, providing a rotational lock between the support structure 33 and the plunger rod 26, yet allowing longitudinal movement between them. The feedback member 18 can be a U-shaped metal bracket for example. The support structure 33 comprises at its distal end 331 a distal support structure 335 having a support recess 337 formed by two lateral arms as seen in FIG. 14. In other words, at the distal end 331 the support structure 335 provides a support for the u-bracket 18 by forming a recess 337 with its lateral arm structures. The u-bracket is received within the two lateral arms of the support structure 335 within the recess 337. Rotational movement of the u-bracket is prevented by the lateral arms. Thus, there is a support added in the slit for the u-bracket and this represents also an angular alignment feature for further assembly steps.

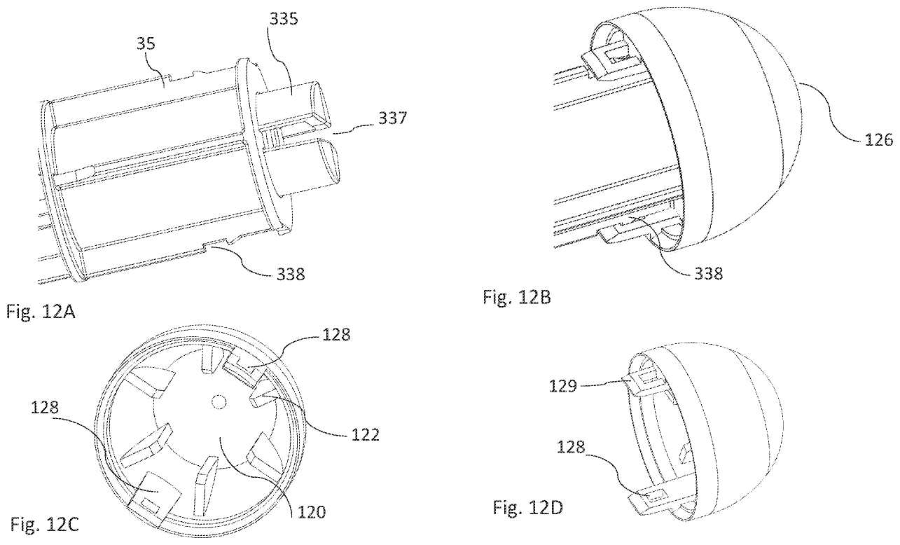

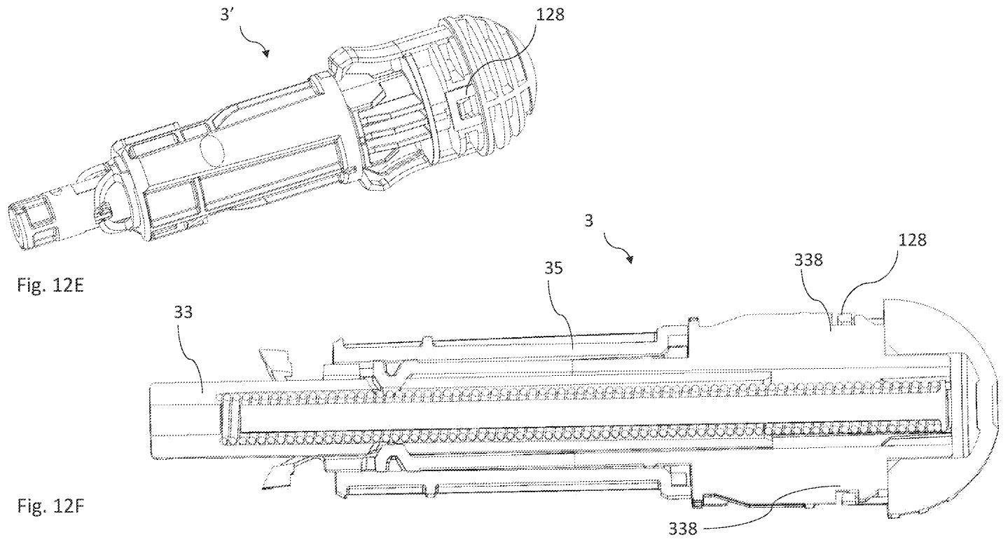

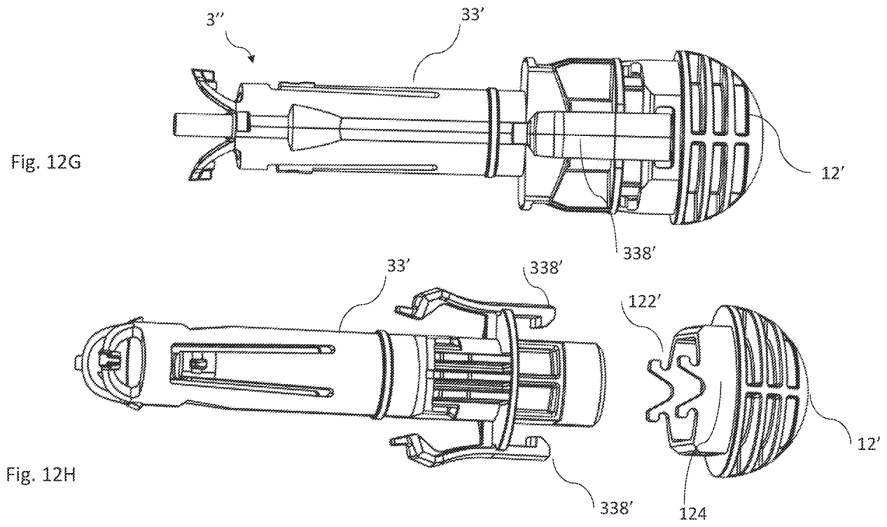

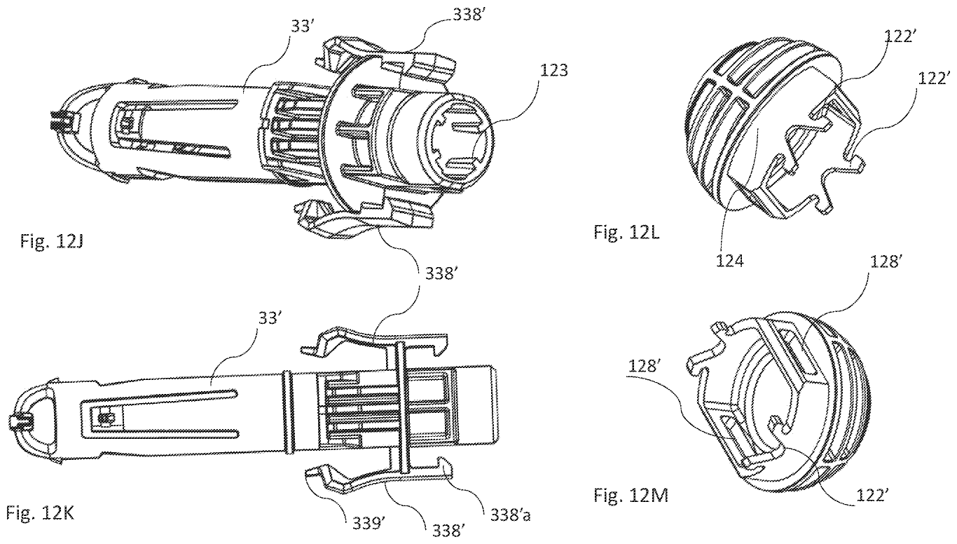

The rear assembly 3, as shown in FIGS. 7C and 12 A-F, further comprises a rear cap 12 which can be assembled in a last step of the manufacturing assemble. The rear cap 12 comprises a proximal opening 120 with one or more engagement members 128 and optionally one or more structural support elements 122.

The rear assembly 3, as shown in FIGS. 7C and 12 A-F, further comprises an actuator 35 and a coupling member 32, or rotator which is rotatably and coaxially arranged on a proximal part of the tubular support structure 33. The actuator 35 is coaxially arranged and fixedly attached to the distal end 1 of the housing 10 preferably by at least one flexible tongue 121 as shown in FIG. 2A, which is engaging with a corresponding distal end portion or rib 301′ arranged as a protrusion on the outer surface of the container holder 30 (see FIG. 3, 8D).

The actuator 35 comprises one or more engagement means 338 configured to engage with the engagement members 128 of the rear cap 12. One amongst other advantages of the rear cap 12, is that during the process of assembling the rear assembly 3, the distal end support structure 335 can securely hold the second resilient 24 member whilst being compressed, and also the feedback member 18 is prevented to accidentally move away from its intended position during and after the assembling. The one or more engagement members 128 when engaging with the engaging means 338 are configured to easily engage with each other during assembling. The engagement members 128 are further designed to tightly fit into the shell of the housing 10, in this way the housing prevents an accidental disengagement or loosening of the engagement members 128 from the engaging means, thus the rear cap 12 is securely hold after assembling. Optionally, the proximal opening 120 of the rear cap may comprise structural support elements 122 further engaging with the actuator 35 or the distal end of the support structure 33. The structural support elements 122 abut on various areas of the actuator 35 or the distal end support structure 335, thus enhancing the engagement of the rear assembly 3. For example, unintended rotation or lateral movement can thus be prevented.

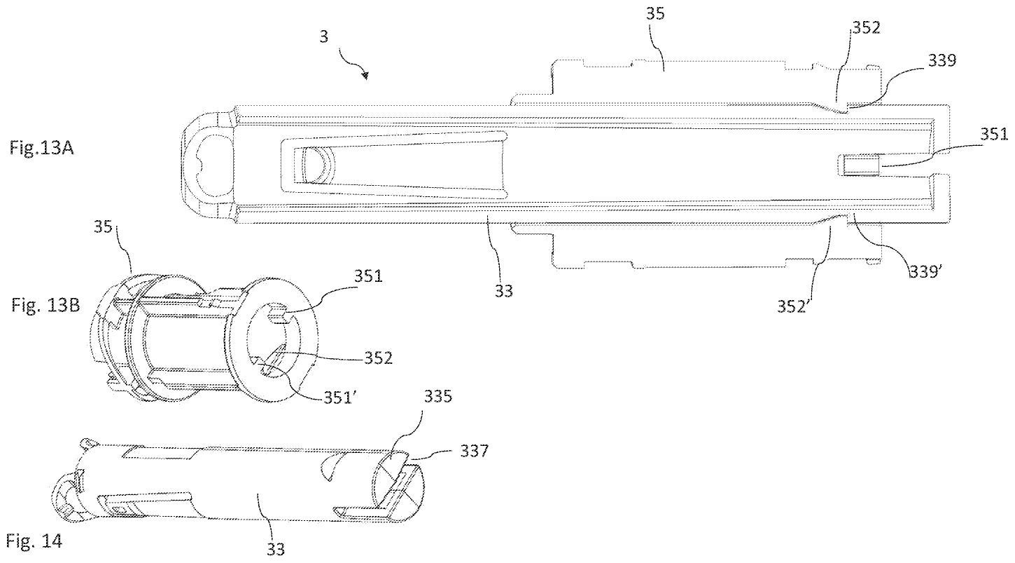

In one embodiment the actuator 35 further comprises engagement members 352, 352′ in form of radially inwardly extending protrusions configured to engage with corresponding recesses 339, 339′ on the outer surface of the support structure 33 as shown in FIG. 13A, FIG. 13B and FIG. 14. When the power pack is being assembled, the support structure 33 is introduced into the actuator 35, the support structure thus slides from the proximal end in a distal direction within the tubular actuator until the engagement members 352, 352′ snap into the recesses 339, 339′ on the surface of the support structure 33. Thereby no further distal movement is possible, the hook like engagement of the engagement member and the recesses do not allow movement into the distal direction.

In one embodiment the actuator 35 further comprises locking elements 351, 351′ as shown in FIG. 13 and FIG. 14, these locking elements are abutting the bottom of the support recess 337 formed by the two lateral arms of the distal support structure 335. These locking elements are radially inwardly extending protrusions of the terminal circumferential edge at the distal side of the actuator. The protrusions have an extension such that they fit within the width of the recess at from two opposing sides of the recess 337. In this example they extend radially only slightly, in order to abut an edge of the terminal section of the recess in order to prevent rotational movement.

In most cases the assembling of medical delivery device or the sub-assembled parts follow a certain procedure and are mostly automated procedures, accordingly during the successive steps of the assembling some partly assembled units have to be securely held or supported, otherwise they may disengage or move out of their intended position. In most cases fully automated assembly machines (FAAM) and high cavity tools are used. For these cases the feedback member support recess 337 provides a fixturing support for the U-bracket 18 while, the second resilient member i.e. the plunger spring and the plunger rod are assembled by force. The slipping of the U-bracket into the rear cap during assembling is an issue being solved by the present disclosure. The feedback member support recess 337 is defined by two or more arms of the distal end support structure 335 between which the u-bracket distal, closed ending can be seated. The distal end support structure 335 thus prevents lateral slipping of the u-bracket and provides a stable support or gripping surface for the FAAM machine for fixing the sub-assembly while the first resilient member is tensioned during assembling.

In an alternative embodiment (not shown in the figures), the support structure 33 does not have a distal end support structure 335 in form of two or more arms, in this case the u-bracket is seated on top of the distal end support structure 335, i.e. the u-bracket is seated on the opposite side of the support structure 35, the inside of the end 335 is an end of dose click seat for the u-bracket to hit and generates a sound indicating that the injection is almost finished. During fully automated machine assembling the sub assembly is seated onto the distal surface of the support structure with the u-bracket and hold such that when the resilient member is tensioned the u-bracket cannot move. Subsequently when the interrelated parts are engaged with each other the rear cap 12 can be mounted on the distal end of the rear assembly 3.

In one example the rear cap has a dome like form with a top surface 126 with a cavity at its proximal opening for receiving the distal end support structure 335 of the of the support structure 33. This cavity may be defined by the support elements 122 or be alternatively an open cavity. The engagement member 128 and engaging means 338 can be a type of snap fit securing means, tapered engaging means, hook engagement means or any other suitable engagement means.

In one example, the engagement member 128 defines a flexible ledge with an oblique proximal edge 129 in order to facilitate the engagement member to slide within the housing 10 during the assembly. The engaging means 338 are arranged on longitudinal protrusions along the outer surface of the actuator 35 and define recesses complementary to the form of the flexible ledges of the engagement member 128. During assembly the housing then forces the flexible ledge into the corresponding recess of the engaging mean 338. The continuous force on the flexible ledge exerted by the housing 10 keeps the rear cap 12 in place.

In yet another example, the engaging means 338 in form of recesses further comprise at their distal end a hook like form such that the hook additionally locks the engagement member 128 such that movement of the rear cap 12 in a distal direction is prevented.

In an alternative embodiment shown in FIG. 12G-FIG. 12M, the rear assembly 3″ further comprises a support structure 33′ with engaging means 338′ in form of radially extending flexible arms configured to snap into corresponding at least one engagement member 128′ in form of a slot, circumferentially arranged on cap 12′. The at least one engagement member 128′ may be arranged on a circumferential rim 124, extending longitudinally from the cap 12′ and having a diameter equal to or inferior of the cap's diameter. The engagement member 128′ can be in form of a recess within the rim 124, the recess can be larger in size than the engaging means 338′, such that slightly different sizes of engaging means 338′ can be fitted within the recess of the engagement member 128′. The engaging means 338′ further comprise at their distal end a hook like form 338′a, such that the hook 338′a additionally locks the engagement member 128′ such that movement of the rear cap 12′ in a distal direction is prevented. The snap fit engagement of the engaging means 338′ with the engagement members 128′ provide an easy to assemble and secure hold of the cap 12′.

The cap 12′ further is provided with structural support elements 122′ configured to engage with guiding ribs 123 which are arranged on the inner side of the tubular support structure 33′. The support elements in this example are at least one pair of flexible legs 122′ able to engage with the support structure 33′ and the guiding ribs 123 such that a loose assembly i.e. any radial or longitudinal movement of the cap 12′ is prevented. One advantage is that tolerances of assembly are mitigated and related side effects like rattling noise due to lose fitting parts can be avoided. The engaging means 338′ optionally further comprise flexible arms 339″ configured to engage with the actuator (not shown in the figures)

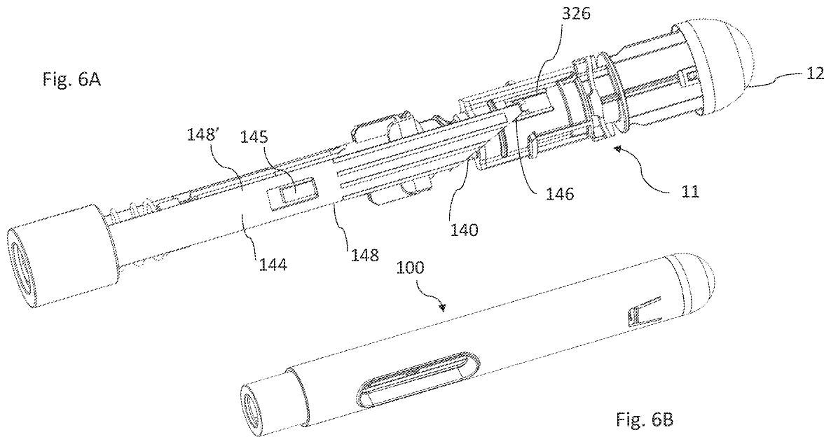

FIG. 1 and FIG. 4A-FIG. 6B show simplified perspective views of the medicament delivery device 100, where FIG. 1 illustrates an initial, non-activated, state of the medicament delivery device 100 having a protective cap 16. An activated state of the medicament delivery device 100, is shown in FIG. 4A without the housing 10 and cap 16 and on FIG. 4B with the housing 10 and the protective cap 16 removed. FIG. 5A shows the penetration and injection state of the medicament delivery device 100, without the housing 10 and FIG. 5B shows it with the housing 10. FIG. 6A shows the medicament delivery device 100 in a final locked state without the housing 10 and FIG. 6B shows the same with the housing. FIGS. 4A, 5A and 6A further show the needle cover 14, which is slidably and coaxially arranged inside the tubular housing 10. The protective cap 16 comprises a distal end surface, abutting with the proximal end surface of the annular contact member 44 of the needle cover 14 such that when the protective cap 16 is manually operated and detached, it allows the needle cover 14 to be placed at the injection site wherein the annular contact member 44 being in contact with the injection site. When the needle cover 14 is further manually operated and pushed against i.e. in direction to the injection site, the needle cover 44 is moved by the force into the housing 10 from its non-activated position to its activated position In order to avoid an accidental activation of the power pack 3, for example during transport if a shock exerts a pressure on the needle cover 14. In these cases the coupling member 32 must be prevented from moving to a released state prematurely. The release state in this example, is related to the movable coupling member 32, which is configured to interact with the support structure for releasing the plunger rod. In the release state the plunger rod is free to move. However, the coupling member 32 is configured to rotate from a first locked position to a second unlocked position.

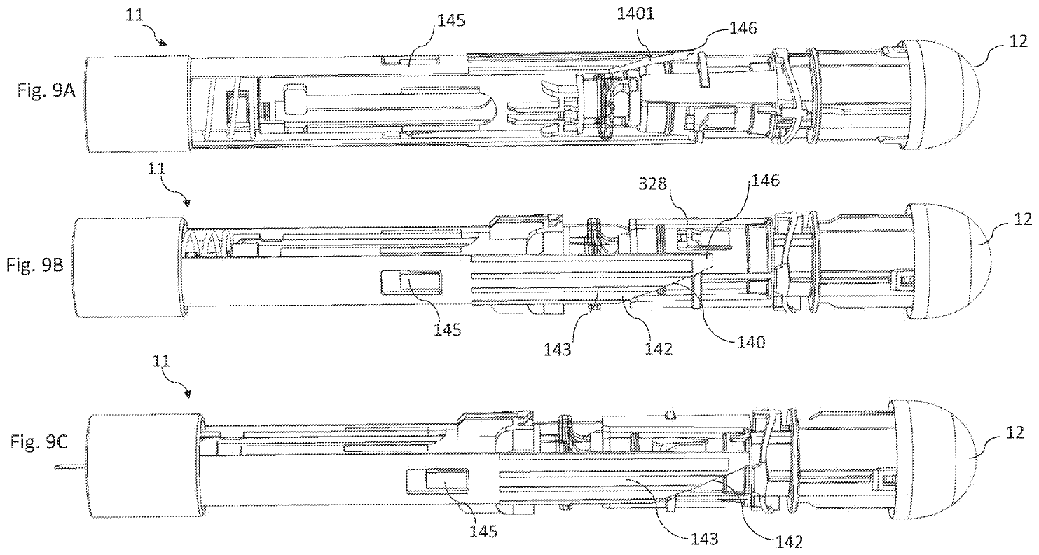

Rotation of the coupling member 32 from the first locked position to the second unlocked position is caused when the needle cover 14 is pushed against an injection site by a user of the device. This causes the needle cover 14 to slide axially in the distal direction relative to the housing 10. The annular contact member 44 having a proximal opening 441 and is extending axially at a predetermined length defining an annular structure. At least one arm 141 extends axially form the annular contact member 44 towards the distal end 1 of the medicament delivery device 100. This elongated structure 141 or needle cover arm is configured to interact with the coupling member 32. This at least one needle cover arm 141 has a triangular or trapezoidal tapering 140 towards a distal end portion 146 as shown in FIGS. 9A, 9B and 9C. One side of the trapezoidal tapering 140 defines a guide surface 1401, configured to cooperate with a protrusion 320, defining an circumferential annular rib on the coupling member 32. The needle cover arm 141 may further be arranged with a radially inwardly extending ledge 145. The medical delivery device 100 is either assembled at the manufacturing site, with the medicament container 20 containing the medicament and being assembled within the device, or it is provided to the medicament provider for a final assembly of the medicament delivery device 100. When the final assembly is realized by the medicament provider, the medicament container 20 must be introduced into the medicament holder 30, which optionally is provided as one item of medicament delivery device and has to be integrated into the medicament delivery device which has to be end assembled. Some parts may be provided in a pre-assembled state. These interacting parts with often irreversibly engaging mechanisms, often have a sensitive or filigree design, which can be damaged during transport, or accidentally exchanged with a similar but different type. Hence when a medicament container 20 is introduced into a damaged sub-assembly and the medicament device end-assembled, the medicament container eventually may not be introduced at all, or securely taken out of the medicament delivery device.

The needle cover arm 141 is provided with a flexible ledge 145 which extends radially outward of the needle cover. The flexible ledge 145 may further comprises a radially inwardly extending protrusion 1451 providing an enlarged stopping surface. During assembly, the flexible ledge 145 may be flexed inwardly, such that it does not extend the needle cover 14 and being slidable into the housing 10. Optionally, guiding ribs, for example 143 align the assembly of the needle cover. When the flexible ledge 145 is provided with a spacing while being slide into the housing, the flexible ledge 145 can then flex outwardly into the spacing. Then the radially inwardly extending protrusion then extends the needle cover arm radially and provides a stopping surface for a protrusion 106a. The enlarged stopping surface 1451 of the flexible ledge 145 engages with a protrusion 106a that is extending radially inwardly from the inner surface of the housing 10. Thus, the flexible ledge 145 prevents the needle cover 14 from further movement in the proximal direction along the longitudinal axis L.

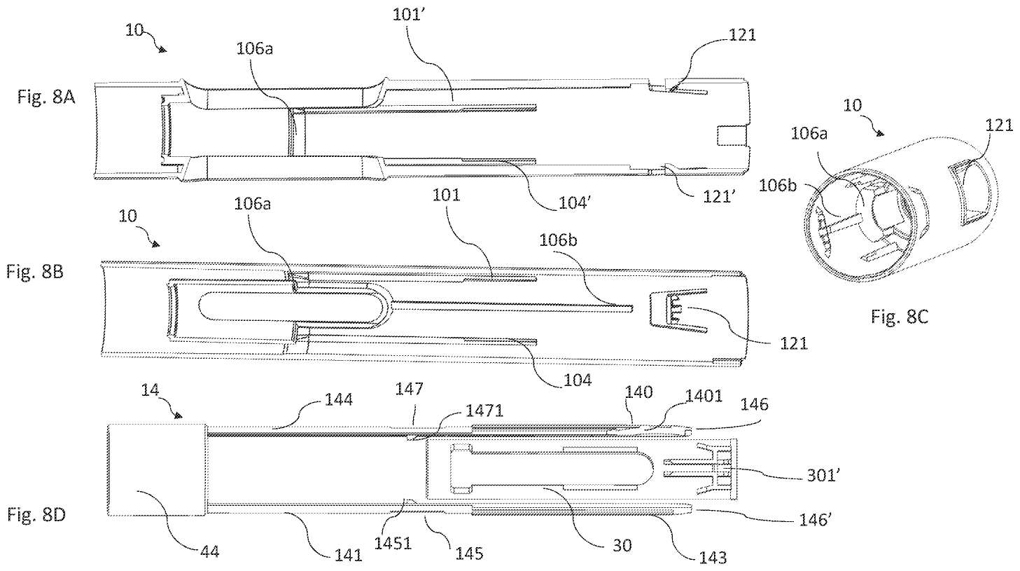

In an alternative embodiment the needle cover arm 141 with the flexible ledge 145 flexes outwardly when the medicament holder is introduced within the tubular body of the needle cover 14. The flexible ledge 145 exerts a force on the medicament holder 30, keeping it in place and acting as a buffer in case of vibrations or shocks occur during handling or use of the medicament delivery device. The flexible ledge 145 may further comprises a radially inwardly extending protrusion 1451 providing an enlarged stopping surface, tolerating less flexing movement of the ledge 145 or a radially smaller medicament holder 30. As is shown in FIG. 8D the protrusion 1451 extends inwardly into the inner space of the needle cover 14 and does not exceed radially the needle cover 14. When the medicament holder 30 being introduced into the needle cover 14, the proximal circular edge of the medicament holder 30 abuts the protrusion 1451 which allows the flexible ledge 145 to bend outwardly while the medicament holder slides further into the needle cover 14. Then the enlarged stopping surface 1451 of the flexible ledge 145 engages with a protrusion 106a that is extending radially inwardly from the inner surface of the housing 10. During assembly the flexible ledge 145 can flex radially outward, such that when abutting the protrusion 106a, the flexible ledge 145 prevents the needle cover 14 from further movement in the proximal direction along the longitudinal axis L. Thus, the needle cover 14 can be axially locked. The external surface of the needle cover arm 141 may further have guide elements e.g. grooves 143 configured to interact with counter acting guide elements on the inner surface of the housing 10 for preventing any rotational movement of the needle cover 14 but allowing a longitudinal axial movement in relation to the housing.

In one embodiment, one or more axially extending protrusions (101, 104) of the inner surface of the housing 10 may be positioned such as to interact with the longitudinal sides (142,142′) of the needle cover arm 141. The axially extending and radially inwardly extending protrusions (101,104) are guiding ribs that are positioned and configured such that the needle cover arm 141 slides along these guiding ribs thus preventing any rotational movement of the needle cover 14.

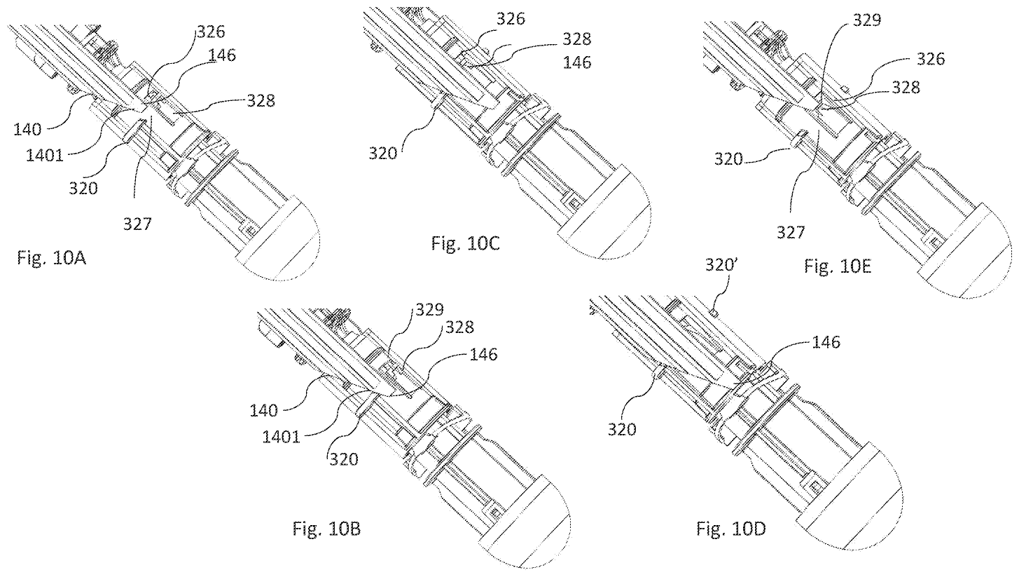

The needle cover arm 141 engages with the coupling member 32 at a distal end portion 146 of the needle cover arms 141. Before the medicament delivery device is activated, the end portion 146 of the needle cover arm 141 is such that it is positioned before a spacing between two protrusions 320, 328 of the coupling member 32 as shown in FIG. 9A and FIG. 10A. The protrusions 320, 328 of the coupling member 32 extend radially outwards from a surface of the coupling member and one of the protrusions 320 defines at least one annular rib of a predefined circumferential length and the other protrusion 328 defines a flexible ledge. The annular rib 320 and the flexible ledge 328, both define a spacing 327 between them on the same radial ring portion, such that the trapezoidal end portion 146 of the needle cover arm 141 can be accommodated within this spacing. As the needle cover moves axially in the distal direction the end portion 146 of the needle cover arm 141 penetrates the spacing 327 as shown in FIG. 9B and FIG. 10B. The distal end portion 146 of the needle cover arm has an oblique or tapered shape on one longitudinal side, such that the triangular shape penetrates the spacing between the annular rib 320 and the flexible ledge 328. The needle cover arm 141 can continue to move in the distal direction until the width of the needle cover arm 141 causes the lateral edges to abut both, the annular rib and the flexible ledge 328 as shown in FIG. 10C.

Subsequently as needle cover 14 and respectively the needle cover arm 141 further penetrates the spacing 327, the needle cover arm 141 thereby slides above the flexible ledge 328 with a longitudinal edge, forcing the flexible ledge to penetrate in to a recess, thus allowing the needle cover arm 141 to slide above the ledge 328. Further, as the lateral edge of the needle cover arm 141 has abutted the edge of the annular rib 320, any further movement of the needle cover 14 translates into a rotational movement of the coupling member 32. In other words, the needle cover 14 activates and rotates the coupling member 32 through the engagement of the annular rib 320 and the needle cover arm 141, as the needle cover arm applies a force on the edge portion of the annual rib with the tapered end portion 146 of the needle cover arm 141. This applied force activates the rotational movement of the coupling member 32.

The movement comes to a halt in a position as shown in FIGS. 9C and 10D optionally abutting a terminal edge of the coupling member, i.e. the tip of the needle cover arm 141 may abut at an annular protrusion or structure. Subsequently after activation, the needle cover 14 is extended to a lock out position, this enables the ledge 328 to flex out of the recess, the ledge comprises a perpendicularly extending structure providing an abutting surface 329 to the terminal edge of the needle cover arm 141. The needle cover arm comes to a halt when abutting the surface 329 of the flexible ledge 328, no further distal movement of the needle cover arm is possible. The needle cover is thus axially locked as shown in FIG. 10E.

In one embodiment the needle cover 14 has two arms 141, 144 extending axially from the annular contact member 44 towards the distal end 1 of the medicament delivery device 100. The external surface of the needle cover arms 141,144 both may further have grooves 143, 143′ (143′ not shown in the figures) configured to interact with the inner surface of the housing 10. One or more axially extending protrusions of the inner surface of the housing may be positioned in the one or more groove 143,143′ configured to slide within in the grooves 143,143′ of the of the needle cover arms 141, 144 thus preventing any rotational movement of the needle cover 14. The needle cover arms are positioned opposite to each other in this example, but other configurations are no excluded. The needle cover arms 141,144 are further provided with flexible ledges 145, 147 which extend radially outward of the needle cover. The flexible ledges 145, 147 may further comprises a radially inwardly extending protrusions 1451,1471 providing enlarged stopping surfaces. During assembly, the flexible ledges 145, 147 may be flexed inwardly, such that they do not extend the needle cover 14 and being slidable into the housing 10. Optionally, guiding ribs, for example 143 align the assembly of the needle cover. When the flexible ledges 145,147 are provided with a spacing while being slide into the housing 10, the flexible ledges 145, 147 can then flex outwardly into the spacing. Then the radially inwardly extending protrusions then extends the needle cover arm radially and provide two stopping surfaces for a circumferential protrusion 106a. The enlarged stopping surface 1451 of the flexible ledges 145,147 engage with the protrusion 106a that is extending radially inwardly from the inner surface of the housing 10. Thus, the flexible ledges 145, 147 prevent the needle cover 14 from further movement in the proximal direction along the longitudinal axis L.

In yet an alternative embodiment the needle cover arms 141, 144 may further both be arranged with a radially inwardly extending ledges 145, 147, exerting a resilient force on the medicament holder 30. The flexible ledges 145 and 147 may further comprise radially inwardly extending protrusions 1451 and 1471 providing an enlarged stopping surface, tolerating less flexing movement of the ledge 145 or 147 or a radially smaller medicament holder 30. As is shown in FIG. 8D the protrusions 1451, 1471 extend inwardly into the inner space of the needle cover 14 and do not exceed radially the needle cover 14. When the medicament holder 30 being introduced into the needle cover 14, the proximal circular edge of the medicament holder 30 abuts the protrusions 1451, 1471 which allows the flexible ledges 145, 147 to bend outwardly while the medicament holder slides further into the needle cover 14. Then the enlarged stopping surfaces 1451, 1471 of the flexible ledges 145, 147 engage with a protrusion 106a that is extending radially inwardly from the inner surface of the housing 10. During assembly the flexible ledges 145, 147 can flex radially outward, such that when abutting the protrusion 106a, the flexible ledges 145, 147 prevent the needle cover 14 from further movement in the proximal direction along the longitudinal axis L. Thus, the needle cover 14 can be axially locked.

In one embodiment, two or more axially extending protrusions (101,101′ 104,104′) of the inner surface of the housing 10 may be positioned such as to interact with the longitudinal sides (142,142′, 148, 148′) of the needle cover arms 141, 144. The axially extending and radially inwardly extending protrusions (142,142′, 148, 148′) are guiding ribs that are positioned and configured such that the needle cover arms both 141, 144 slide along these guiding ribs thus preventing any rotational movement of the needle cover 14.

The needle cover arms 141, 144 engage with the coupling member 32 at a distal end portion 146, 146′ of the needle cover arms 141, 144. Before the medicament delivery device is activated, the end portions 146, 146′ of the needle cover arms 141, 144 are such that they are respectively positioned before a spacing between two respective protrusions 320, 328 and 320′,328′ (328′ not shown in the figures) of the coupling member 32 as shown in FIG. 9A and FIG. 10A. The protrusions 320, 328, and 320′,328′ of the coupling member 32 extend radially outwards from a surface of the coupling member and two of the protrusions 320 and 320′ define two annular ribs on the same circumferential ring and are of a predefined circumferential length. The other protrusions 328 and 328′ define flexible ledges. The annular ribs 320, 320′ and the flexible ledges 328,328′ both pairwise define respective spacings 327, 327′ (327′ is not shown in the figures) between them on the same radial ring portion, such that the trapezoidal end portions 146, 146′ of the respective needle cover arm 141 and 144 can be accommodated within this spacing. As the needle cover moves axially in the distal direction, the end portions 146, 146′ of the needle cover arms 141, 144 penetrate the respective spacings 327, 327′ (327′ is not shown in the figures) as shown in FIG. 9B and FIG. 10B. The distal end portions of the needle cover arms have both a triangular or trapezoidal shape on one longitudinal side, such that the triangular shapes penetrate the respective spacings between the annular ribs 320, 320′ and the corresponding flexible ledges 328, 328′. The needle cover arms 141, 144 can continue to move in the distal direction until the width of the needle cover arms 141, 144 causes the respective lateral edges to abut both, the annular ribs and the flexible ledges 328, 328′ as shown in FIG. 10C.

Subsequently as the needle cover 14 and respectively the needle cover arms 141, 144 further penetrates the spacing 327,327′ the needle cover arms 141, 144′ thereby slide above the flexible ledges 328, 328′ with their respective longitudinal edges, forcing the flexible ledges to penetrate in to corresponding recesses, thus allowing the needle cover arms 141, 144′ to slide above the ledges 328, 328′. Further, as the lateral edges of the needle cover arms 141, 144 have abutted the edges of the annular ribs 320,320′ any further movement of the needle cover 14 translates into a rotational movement of the coupling member 32. In other words, the needle cover 14 activates and rotates the coupling member 32 through the engagement of the annual ribs 320, 320′ with the needle cover arms 141,144 as the needle cover arms apply a force on the edge portions of the annual ribs with the trapezoidal end portions 146,146′ of the needle cover arms 141, 144. This applied force activates the rotational movement of the coupling member 32.

The movement comes to a halt in a position as shown in FIGS. 9C and 10D optionally abutting terminal edges of the coupling member, i.e. the tips of the needle cover arms 141, 144 may abut at an annular protrusion or structure. Subsequently after activation, the needle cover 14 is extended to a lock out position, this enables the ledges 328, 328′ (328′ is not shown in the figures) to flex out of the recesses 326, 326′ (326′ is not shown in the figures), the ledges comprise respectively a perpendicularly extending structure providing an abutting surface 329, 329′ (329′ not shown in the figures) to the terminal edges of the needle cover arms 141, 144. The needle cover arms come to a halt when abutting the surfaces 329, 329′ of the flexible ledges 328, 328,′ no further distal movement of the needle cover arms is possible. The needle cover 14 is thus axially locked as shown in FIG. 10E.

As mentioned above, the medical delivery device 100 is either assembled at the manufacturing site, with the medicament container 20 containing the medicament and being assembled within the device. Thus, the medicament delivery device 100 is ready to be used when leaving the manufacturing site, or the medicament delivery device 100 is provided to the medicament provider for a final assembly of the medicament delivery device 100. In this second case, it is more practical and advantageous for the medicament provider to receive pre-assemble parts if possible. Some of the parts of the medicament delivery device 100, for example the power pack 3, can be provided in a pre-assembled state. The advantage of receiving a pre-assembled power pack may be acknowledged seeing FIG. 7C showing an exploded view of the exemplary power pack 3 with all its individual parts. Some challenges with pre-assembled parts like the power pack 3, can be an accidental activation for example. Accordingly, a suitable transport lock mechanism is provided during the transport, preventing an accidental activation of the power pack 3.

One improved transport lock mechanism is disclosed in the following exemplary embodiment, in which the coupling member 32 is rotationally movable around a longitudinal axis L, and axially fixed, relative to the actuator 35, which further comprises a locking member 321 that is provided to prevent the coupling member 32 from moving relative to the actuator 35. The locking member 321 is axially movable, and rotationally locked relative to the actuator 35 and relative to the coupling member 32. However, the locking member 321 is configured to interact with the coupling member 32, such that when the locking member 321 is in the first state, the locking member 321 engages with a mating member 322, causing the coupling member 32 to be immobilized by the locking member 321. When the locking member 321 is moved to the second state, the control member 32 is released by the locking member 321.

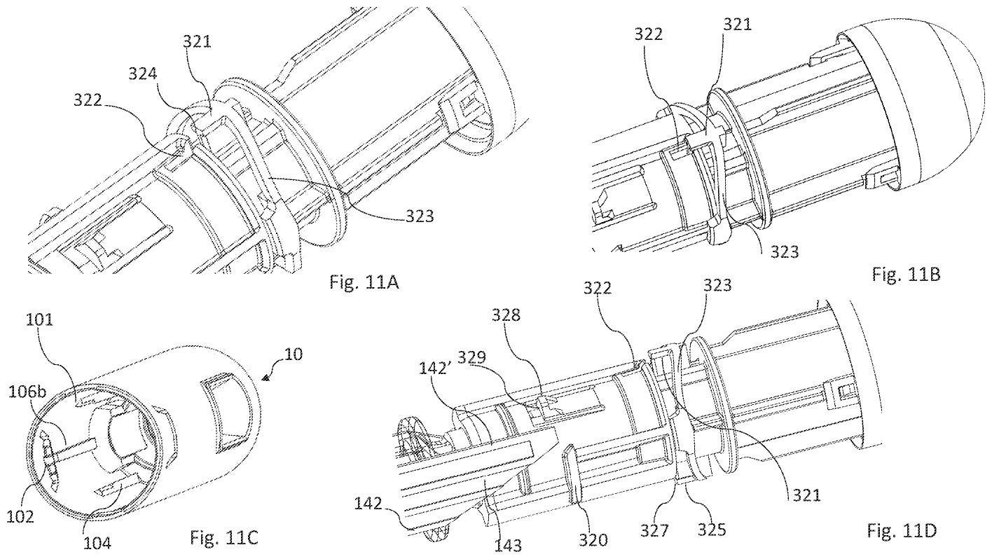

The locking member 321 may comprise a support member 323 which may be an annular or tubular member arranged around the actuator 35, the annular support member 323 is configured as an axially member, which may be a flexible integrated unitary part of the actuator 35, such that the support member 323 is prevented from axial or distal displacement. Alternatively, the annular support member 323 may be configured as a rigid axially member, being fixedly attached to the actuator 35 at least one portion of the annular structure, preventing axial and distal displacement of the annular support member 323. An integrated locking member means fewer components to assemble and reduces the tolerance chain, which leads to a more robust and reliable device. The annular support member 323 is this case configured to be bendable in the distal direction at the annular portion, where the at least one locking member 321 is located. The resilience of the annular support member 323 allows to re-engage the locking member 321 with the corresponding mating member 322 should they be accidentally moved or disengaged during handling or transport of the power pack. In other words, 323 is a flexible support member that will hold locking member 321 in the front position in the slot of the mating member 322 of the coupling member 32 during transport as sub-assembly as shown in FIG. 11B. moved backwards After final assembly the support member 323 moves backwards, i.e. in the distal direction. However, the support member 323 may still be in a tensioned state thus upon removal of the rear assembly 3 the support member 323 moves the locking member 321 again in the slot of in the mating member 322, and thus holds the locking member 321 locked.

The mating member 322 is configured to be engageable with the coupling member 32, the mating member 322 and the support member 323 are shown in FIG. 11A in a disengaged state and in FIG. 11C in an engaged state. A certain threshold force is required to displace the support member 323 axially in order to disengage the mating member 322 from the corresponding locking member 321. During the final assembling of the medical delivery device 100 the required force for disengaging the locking member 321 is provided upon insertion of the power pack 3 in the housing 10. At this stage the locking member 321 abuts a longitudinally elongated protrusion 106b located at the inner surface of the of the housing 10. The elongated protrusion 106b is configured to have a pre-defined length and position within the housing 10 in order to provide a contact member for the locking member 321. During final assembly, when the housing is provided and the power pack 3 is introduced into the housing 10, as the locking member 321 is abutting the distal end portion of the elongated protrusion 106b, the locking member 321 is axially displaced by the elongated protrusion 106b towards the distal direction causing the locking member to disengage from the mating member 322, thus allowing the coupling member 32 rotational movement. After the final assembling the elongated protrusion 106b may further keep the transport lock 321 compressed towards the distal direction, in other words the distal end portion of the elongated protrusion 106b may remain in contact with the abutting surface 324 of the of the locking member 321, thus keeping the transport lock mechanism disengaged after assembling. The mating member 322 is configured to receive the locking member and may have a shape corresponding to the reciprocal shape of the locking member 321 such that the locking member fits exactly into the space of the mating member 322. Alternatively, the mating member can be configured to receive a variety of locking members of different width or length. The shape of the mating member 322 may be tapered for this purpose.

In one embodiment, in which the coupling member 32 is rotationally movable around a longitudinal axis, and axially fixed, relative to the actuator 35, which further comprises at least two locking members 321 and 325 which are provided to prevent the coupling member 32 from moving relative to the actuator 35. The locking members 321, 325 are axially movable, and rotationally locked relative to the actuator 35 and relative to the coupling member 32. However, the locking members 321, 325 are configured to interact with the coupling member 32, such that when the locking members 321, 325 are in the first state, the locking members 321, 325 engage with mating members 322, 322′ causing the coupling member 32 to be immobilized by the locking members 321, 325. When the locking member 321, 325 are moved to the second state, the control member 32 is released by the locking members 321, 325.

The locking members 321, 325 may comprise a support member 323 which may be an annular or tubular member arranged around the actuator 35, the annular support member 323 is configured as an axially member, which may be a compressible integrated unitary part of the actuator 35, such that the support member 323 is prevented from axial or distal displacement. Alternatively, the annual support member 323 may be configured as a rigid axially member, being fixedly attached to the actuator 35 at least one portion of the annular structure, preventing axial and distal displacement of the annular support member 323. An integrated locking member means fewer components to assemble and reduces the tolerance chain, which leads to a more robust and reliable device. The annular support member 323 is this case configured to be bendable in the distal direction at the annual portion, where the at least two locking members 321, 325 are located. The mating members 322, 322′ of the support member 323 are engaged with the coupling member 32 in FIG. 11A one mating member is shown in an disengaged state with the corresponding locking member, in FIG. 11C one mating member is shown engaged with one corresponding locking member. A certain threshold force is required to displace the support member 323 axially in order to disengage the mating member 322 from the corresponding locking members 321, 325. During the final assembling of the medical delivery device 100 the required force for disengaging the locking members 321, 325 is provided upon insertion of the power pack 3 in the housing 10. At this stage the locking members 321, 325 abut a longitudinally elongated protrusions 106b,106b′ located at the inner surface of the of the housing 10. The elongated protrusions 106b,106b′ are configured to have a pre-defined length and position within the housing 10 in order to provide a respective contact member for the locking members 321, 325. During final assembly, when the housing is provided and the power pack 3 is introduced into the housing 10, the locking members 321, 325 are abutting the distal end portions of the respective elongated protrusions 106b,106b′ thus the locking members 321, 325 are axially displaced by the elongated protrusions 106b, 106b′ towards the distal direction causing the locking member to disengage from the mating members 322, 322′ thus allowing the coupling member 32 rotational movement. After the final assembly the elongated protrusions 106b, 106b′ may further keep the transport lock 321, 325 compressed by the elongated protrusion 106b, 106b′ towards the distal direction, in other words the distal end portions of the elongated protrusions 106b, 106b′ may remain in contact with the abutting surface 324, 324′ (324′ not shown in the figures) of the of the locking members 321, 325 thus, keeping the transport lock mechanism after assembly disengaged in place. The mating members 322, 322′ are configured to receive the locking members and may have a shape corresponding to the reciprocal shape of the locking members 321, 325 such that the locking members fit exactly into the space of the mating members 322, 322′. Alternatively, the mating members can be configured to receive a variety of locking members of different width or length. The shape of the mating member 322, 322′ may be tapered for this purpose. In one embodiment the mating members 322 and 322′ may have different shapes. The locking member 321 may be identical in shape and form to the locking member 325. Alternatively, the locking member 321 may have a different shape and form of the locking member 325. The locking member 321 and 325 may be configured to have the same of a different force required to disengage them from the respective mating members 322, 322′. One advantage of two locking members, is that one locking member may still provide a transport lock even if the other locking member may have a default or be broken.

After assembly, the medicament delivery device would typically be provided.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

1-15. (canceled)

16: A medicament delivery device comprising:
an actuation mechanism configured to act on a medicament container for expelling a medicament comprising:
a rear assembly comprises a transport lock mechanism, a support and holding mechanism for a feedback member and plunger rod;
an activation member;
an elongated housing having a proximal end and an opposite distal end, accommodating the rear assembly, the medicament container, the actuation mechanism and the activation member, wherein the housing further comprises guiding members able to interact with the transport lock mechanism of the rear assembly
a rear cap;
wherein the biased activation member is axially slidable with respect to the housing from a proximal extended position to a retracted position and operably connected to a tubular coupling member, rotating the coupling member from an initial non-activated rotational position to an activated rotational position when the activation member slides from the extended position to the retracted position;
and wherein the coupling member further comprises a blocking element arranged to block the activation member in a final position after expelling of the medicament.

17: The medicament delivery device according to claim 16, wherein the activation member further comprises at least one arm with a flexible radially inwardly extending ledge arranged in a recess of the arm of the activation member, wherein the inwardly extending ledge, further comprises a radially inwardly extending protrusion providing an enlarged stopping surface.

18: The medicament delivery device according to claim 16, wherein the transport lock mechanism, comprises a radially outwardly extending locking member arranged on an actuator and configured to interact with a mating member being a part of the coupling member and configured to engage with the locking member such that the coupling member is prevented of axial movement.

19: The medicament delivery device according to claim 18, wherein the transport lock mechanism when in a first state, the mating member mates with the corresponding locking member of the coupling member such that rotation of the coupling member relative to the actuator and the locking member is prevented; in a second state, the guiding members of the housing abutting a surface of the transport lock mechanism and during final assembly pushes the locking member out of the engagement with the corresponding mating member.

20: The medicament delivery device according to claim 16, wherein the housing comprises at least one axially extending protrusion being guiding members positioned such that at least one arm of the activation member is axially guided and a radial movement beyond the abutment with the protrusion is prevented by the protrusion.

21: The medicament delivery device according to claim 16, when the actuation mechanism further comprises a resilient member and wherein the at least one arm of the activation member comprises a tapered distal portion wherein the tapered distal portion defines a guide surface at one of the longitudinal sides, wherein the guide surface is configured to cooperate with the coupling member, when the resilient member biases the activation member towards the activated rotational position.

22: The medicament delivery device according to claim 21, wherein rotating the coupling member from the initial non-activated rotational position to the activated rotational position comprises the guide surface abutting a radially outwards extending protrusion optionally on a circumferential annular rib on the coupling member, wherein the protrusion optionally comprises a to the guide surface complementary oblique contact surface.

23: The medicament delivery device according to claim 16, wherein the coupling member further comprises a protrusion defining a flexible ledge, being compressible radially inwards, when one of the longitudinal sides of the activation member arm slides above the flexible ledge.

24: The medicament delivery device according to claim 23, wherein the flexible ledge is a blocking element which flexes radially outwards when a first resilient member of the rear assembly actuates the expelling of the medicament and thereby displaces the activation member axially in a proximal direction, wherein the flexible ledge flexes radially outward when a distal end portion of the activation member arm disengages and releases the flexible ledge; wherein the flexible ledge then provides an abutting surface for the distal end portion blocking a distal movement of the activation member.

25: The medicament delivery device according to claim 16, wherein the support and holding mechanism for the feedback member and plunger rod comprising the actuator having one or more engagement members configured to engage with engagement members of the rear cap.

26: The medicament delivery device according to claim 25 wherein the engagement member has a flexible ledge with an oblique proximal edge configured to guide the flexible ledge into the housing during assemble.

27: The medicament delivery device according to claim 26, wherein the engagement means define a recess on at least one longitudinal protrusion arranged along the outer surface of the actuator, when the rear cap being assembled, the engagement member has a complementary fitting shape to fit into the recess and securely hold the cap in place, wherein the recess optionally has a hook for holding the engagement member of the cap locked such that once assembled the cap is not removable.

28: The medicament delivery device according to claim 25 wherein the engagement member has flexible arms further comprising a hook from configured to the engage with the engagement member

29: The medicament delivery device according to claim 27, wherein the actuator further comprises at least one locking element in form of a radially inwardly extending protrusion along the inner surface of the actuator, the locking element is abutting the bottom of a supporting recess formed by two lateral arms of a distal end portion of the support structure, wherein the locking element engages with by fitting within the width of the recess in order to prevent the feedback element to move distally and prevent a rotational movement of the support structure.

30: The medicament delivery device according to claim 27, wherein the actuator further comprises at least one engagement member in form of radially inwardly extending protrusion configured to engage with a corresponding recess arranged on the outer surface of the support structure, during assembly the support structure slides from the proximal end in a distal direction within the tubular actuator until the engagement member flexes into the corresponding recesses preventing any further movement of the support structure in a distal direction.

31: A medicament delivery device comprising:
an actuation mechanism configured to act on a medicament container for expelling a medicament comprising:
a rear assembly comprises a transport lock mechanism, a support and holding mechanism for a feedback member and plunger rod; and
an activation member that is biased proximally and comprises two distally extending arms each having a radially inwardly extending protrusion,
an elongated housing having a proximal end and an opposite distal end, where the housing accommodates the rear assembly, the medicament container, the actuation mechanism and the activation member and where the housing further comprises guiding members that interact with the transport lock mechanism of the rear assembly, and
a rear cap;
wherein the biased activation member is axially slidable with respect to the housing from a proximal extended position to a retracted position and operably connected to a tubular coupling member, where rotating the coupling member from an initial non-activated rotational position to an activated rotational position when the activation member slides from the extended position to the retracted position,
wherein the coupling member further comprises a blocking element arranged to block the activation member in a final position after expelling of the medicament, and
wherein the transport lock mechanism comprises a radially outwardly extending locking member on an actuator that engages with a mating member on the coupling member to prevent the coupling member from moving axially.

32: The medicament delivery device of claim 31, wherein when the transport lock mechanism is in a first state, the mating member and the locking member are engaged such that rotation of the coupling member relative to the actuator is prevented and when in a second state, the guiding members of the housing are abutting a surface of the transport lock mechanism such that during final assembly the guiding members push the locking member out of the engagement with the mating member.

33: The medicament delivery device of claim 31, wherein a resilient member is engaged with the activation member.

34: The medicament delivery device of claim 31, where each arm terminates in a tapered distal portion comprising a guide surface that engages with the coupling member, when the activation member moves axially towards the retracted position.

35: The medicament delivery device of claim 31, wherein an outer surface of the coupling member comprises a protrusion defining a flexible ledge that is compressed radially inwards when one of the radially inwardly extending protrusions on the arms slides up and over the flexible ledge.
